(19) United States
(12) Patent Application Publication
(10) Pub. No.: US 2025/0018357 A1
(43) Pub. Date: Jan. 16, 2025

(54) REACTOR FOR NON-OXIDATIVE DIRECT CONVERSION OF METHANE AND METHOD FOR PREPARING ETHYLENE AND AROMATIC COMPOUND BY USING SAME

(71) Applicant: KOREA RESEARCH INSTITUTE OF CHEMICAL TECHNOLOGY, Daejeon (KR)

(72) Inventors: Yong Tae KIM, Daejeon (KR); Sung Woo LEE, Daejeon (KR); Jungho SHIN, Daejeon (KR); Seung Ju HAN, Daejeon (KR); Tsegay Gebrekidan GEBREYOHANNES, Daejeon (KR)

(73) Assignee: KOREA RESEARCH INSTITUTE OF CHEMICAL TECHNOLOGY, Daejeon (KR)

(21) Appl. No.: 18/687,504

(22) PCT Filed: Aug. 2, 2022
(86) PCT No.: PCT/KR2022/011421

§ 371 Date: Feb. 28, 2024

(30) Foreign Application Priority Data
Sep. 8, 2021 (KR) 10-2021-0119675

(51) Int. Cl. B01J 19/02; B01J 19/24; C07C 2/74

(52) U.S. Cl. CPC B01J 19/02; B01J 19/2415; C07C 2/74; B01J 2219/0227

(57) ABSTRACT

The present invention relates to a nonoxidative methane direct converting reactor and a preparation method of ethylene and aromatic compound using the same and, more specifically, to a nonoxidative methane direct converting reactor and a preparation method of ethylene and aromatic compound using the same, wherein the reactor maximizes a catalytic reaction rate, minimizes cokes production, and provides a high conversion rate of methane and a high yield of ethylene and an aromatic compound even at a low hydrogen supply ratio in the production of ethylene and an aromatic compound from methane.

### TECHNICAL FIELD

The present invention relates to a nonoxidative methane direct converting reactor and a preparation method of ethylene and aromatic compound using the same and, more specifically, to a nonoxidative methane direct converting reactor capable of preparing ethylene and aromatic compound by directly converting methane which is a main component of natural gas, under an anaerobic or oxygen-free atmosphere, and a preparation method of ethylene and aromatic compound using the same.

### BACKGROUND TECHNOLOGY

Recently, efforts have been made to convert methane (CH4), which may be obtained from natural gas, sailing gas, etc., into high value-added products such as transportation fuel or chemical raw materials.

Representative examples of the high value-added products that may be obtained from methane include olefins (ethylene, propylene, butylene, etc.) and aromatic compound, and it is known that the most feasible technologies are a methanol to olefin (MTO) technology in which a light olefin is prepared by passing a synthesis gas (H2+CO) obtained through methane reforming through methanol and a fischer-tropsch to olefin (FTO) technology in which a light olefin is directly produced from the synthesis gas.

However, in the case of the technology of producing high value-added products via the synthesis gas, hydrogen (H2) or carbon monoxide (CO) is additionally required to remove oxygen atoms from carbon monoxide (CO), which results in deterioration of utilization efficiency of hydrogen atoms or carbon atoms in the entire process.

Therefore, there is a need for a new technology capable of directly converting methane into high value-added products without passing through synthesis gas. In order to directly convert methane into a high-value product, a C—H bond (434 kJ/mol) strongly formed in the methane is cut to activate the methane, and from this point of view, studies on an Oxidative Coupling of Methane (OCM) technology for activating methane using oxygen have been actively conducted. However, in the OCM reaction, a large amount of thermodynamically stable H2O and CO2 are formed due to the intense reactivity of O2, and thus, it is still pointed out that the utilization efficiency of hydrogen atoms or carbon atoms is lowered.

In order to solve such problems, technologies for preparing ethylene, aromatic compound, and the like by direct conversion of methane under anaerobic or oxygen-free conditions have been recently developed, but due to low reactivity of methane, the technologies are being conducted at high temperature and high pressure, and thus, development of suitable reactors and catalysts is essential. However, according to the research results, the problem of rapid decrease in catalyst activity due to the deposition of carbon (cokes) of the catalyst under the conditions of high temperature and high pressure is highlighted as a core issue (see non-patent documents 0001 and 0002).

Thus, U.S. Pat. No. 4,424,401 discloses a method of diluting acetylene with inert gas, water, hydrogen, methane, and alcohol in the presence of zeolite catalyst ZSM-5 to aromatize the mixture into a hydrocarbon mixture, and U.S. Pat. No. 8,013,196 discloses a method of thermally converting methane-containing supply into acetylene-containing supply by pyrolysis, and hydrogenating the converted acetylene-containing supply to prepare ethylene.

However, as described in these prior documents, the aromatization of methane or acetylene on zeolite or other catalysts also has a problem in that the performance of the catalyst is exhibited for a very short period of time and is rapidly inactivated due to the accumulation of coke fragments and rapid polymerization of acetylene. In addition, high content of other by-products derived from acetylene conversion were formed.

In particular, known methods of producing ethylene, aromatic compound in methane-containing supply have shown several disadvantages, such as catalyst inactivation, excessive hydrogenation, formation of green oil or carbon, problems of temperature overheating, or low production per unit volume of the reactor.

Therefore, there is a need in the art to develop technology for an improved method and a reactor, which enables more efficient and stable preparation of aromatic compound and ethylene from methane.

### PRIOR ART DOCUMENT

### Patent Document

(Patent Document 1) U.S. Patent Registration No. 4424401 (Registration Date: 1984 Jan. 3)
(Patent Document 2) U.S. Pat. No. 8,013,196 (Registration Date: 2011 Sep. 6)

### Non-Patent Document

(Non-patent document 1) X, Guo et al., Direct, Nonoxidative Conversion of Methane to Ethylene, Aromatics, and Hydrogen, Science, 344, 2014, 616˜619
(Non-patent document 2) Mann Sakbodin et al., Hydrogen-Permeable Tubular Membrane Reactor: Promoting Conversion and Product Selectivity for Non-Oxidative Activation of Methane over an FeVSiO2 Catalyst, Angew. Chem. 2016, 128, 16383˜16386

### DETAILED DESCRIPTION OF THE INVENTION

### Technical Problems

The present invention has been made in an effort to provide a nonoxidative methane direct converting reactor capable of maximizing a catalytic reaction rate, minimizing the preparation of cokes, and providing a high conversion rate of methane and a high yield of ethylene and an aromatic compound in the preparation of ethylene and an aromatic compound from methane, and a preparation method of ethylene and aromatic compound using the same.

### Technical Solving Method

In order to achieve the above objects, an embodiment of the present invention provides a nonoxidative methane direct converting reactor, comprising: an inlet for introducing a methane-containing supply; a reaction unit for generating a ethylene and aromatic compound-containing product by reacting the methane-containing supply introduced from the inlet; and an outlet for discharging the ethylene and aromatic compound-containing product generated from the reaction unit, wherein the reaction unit is divided into a first reaction zone unit for generating acetylene by the reaction of the methane-containing supply introduced from the inlet and a second reaction zone unit for generating ethylene and an aromatic compound by hydrogenating the acetylene generated from the first reaction zone unit, a carbon layer is formed on inner circumferential surfaces of the reactors of the first reaction zone unit and the second reaction zone unit, and a metal compound supported on the carbon layer is used in the second reaction zone unit on which the carbon layer is formed.

In a preferred embodiment of the present invention, the carbon layer may be a cokes layer formed by a nonoxidative methane direct conversion reaction as a reactor.

In a preferred embodiment of the present invention, the carbon layer may have methane conversion activation energy of 300 kJ/mol to 380 kJ/mol.

According to a preferred embodiment of the present invention, the metal compound may include at least one selected from the group consisting of palladium, platinum, iridium, rhodium, iron, chromium, nickel, molybdenum, gold, silver, copper, and indium.

In a preferred embodiment of the present invention, the methane-containing supply may include methane and hydrogen, and a volume ratio of hydrogen against methane (H2/CH4) may be 1.1˜5.0.

In a preferred embodiment of the present invention, the reaction of the first reaction zone unit may be conducted at temperatures ranging from 30° C. to 900° C. and pressures below 10 bar.

In a preferred embodiment of the present invention, the hydrogenation of the second reaction zone unit may be performed at 30° C. to 900° C. and 10 bar or less.

In a preferred embodiment of the present invention, the first reaction zone unit gas hourly space velocity (GHSV) may be 1,000 h⁻¹ to 6,000 h⁻¹.

In a preferred embodiment of the present invention, the second reaction zone unit weight hourly space velocity (WHSV) may be 1.00×104 mlg_{Pd}h⁻¹ to 1.00×109 mlg_{Pd}h⁻¹.

Another embodiment of the present invention provides a preparation method of ethylene and aromatic compound, the method comprising preparing ethylene and an aromatic compound from methane using the nonoxidative methane direct converting reactor.

### Effects of the Invention

According to the present invention, in the preparation of ethylene and an aromatic compound from methane, it is possible to maximize the catalytic reaction rate, minimize the production of cokes, and provide a high conversion rate of methane and a high yield of ethylene and an aromatic compound even at a low hydrogen supply rate.

### BRIEF DESCRIPTION OF DRAWINGS

FIG. 1 is a longitudinal cross-sectional view of a nonoxidative methane direct converting reactor according to an embodiment of the present invention.

FIG. 2 is a schematic diagram of a nonoxidative methane direct converting reactor according to an exemplary embodiment of the present invention with compartments divided at predetermined intervals (i), and SEM images (ii) of carbon layers corresponding to the reactor compartment locations.

FIG. 3 is a graph showing results of measuring activation energy of a carbon layer corresponding to the reactor compartment position of FIG. 2.

FIG. 4 is a graph showing the consumption rate of acetylene (a) and the ratio of acetylene to acetyl (b) according to the reaction time in the reactors of Example 3 and Comparative Example 1 of the present invention.

FIG. 5 is a graph showing the results of measuring the conversion rate of methane and the selectivity of product (a) and the distribution and yield of C2 product (b) according to the reaction time in the reactor of Example 3 of the present invention.

### CODE DESCRIPTION

100: Nonoxidative methane direct converting reactor
110: Inlet
111: Carbon layer
120: Reaction unit
121: First reaction zone unit
122: Second reaction zone unit
123: Metal compound
130: Outlet

### MODE FOR THE INVENTION

Unless defined otherwise, all technical and scientific terms used herein have the same meaning as commonly understood by those skilled in the art to which the present invention pertains. In general, the nomenclature used herein is well known and commonly used in the art.

When a part “comprises” a certain feature element in the present specification, this means that other feature elements may be further included, rather than excluding other elements, unless specifically stated otherwise.

The terms “including,” “comprising,” or “having” described herein refer to the presence of a feature, a numerical value, a step, an operation, a component, a part, or a combination thereof described herein, and do not exclude the possibility that other features, numerical values, steps, operations, components, parts, or combinations thereof not mentioned may be present or added.

Throughout the present specification, the term “reaction unit” or “reaction zone” means a space within a reactor in which a reactant is reacted, the term “inside” and “inner part” means a reactor in a radial center direction of a circle which is a cross-section vertically cut from the direction of gravity, and the term “outside” or “outer part” means a reactor in a radial circumferential direction of a circle which is a cross-section vertically cut from the direction of gravity.

In addition, throughout the present specification, the names of features are divided into first, second, and the like to clearly describe the features, and the names of the features are divided into the same relationship, and the description below is not necessarily limited to the order.

According to one aspect, the present invention relates to a nonoxidative methane direct converting reactor, comprising: an inlet unit which introduces a methane-containing supply; a reaction unit which reacts the methane-containing supply introduced from the inlet unit to produce a ethylene and aromatic compound-containing product; and an outlet unit which discharges the ethylene and aromatic compound-containing product produced from the reaction unit, wherein the reaction unit is divided into a first reaction zone unit which produces acetylene by the reaction of the methane-containing supply introduced from the inlet unit, and a second reaction zone unit which produces ethylene and an aromatic compound by hydrogenating the acetylene produced from the first reaction zone unit, a carbon layer is formed on the inner circumferential surfaces of the reactors of the first reaction zone unit and the second reaction zone unit, and a metal compound supported on the carbon layer is used in the second reaction zone unit on which the carbon layer is formed.

More specifically, in the preparation of ethylene and aromatic compound from methane, the main factors that induce cokes formation are the surface of the catalyst cluster loaded into the reactor, the stagnant flow of the fluid, the reactor material, the reactor surface roughness, etc.

According to the present invention, the reaction unit of the nonoxidative methane direct converting reactor is divided into a first reaction zone unit and a second reaction zone unit; the inner wall of the reactor is stabilized by forming a carbon layer on the inner circumferential surface of the reactor in the first reaction zone unit and the second reaction zone unit; the production of cokes is minimized while maximizing the catalytic reaction rate by supporting the metal compound on the carbon layer in the second reaction zone unit; and the high conversion rate of methane and the high yield of ethylene and aromatic compound can be provided even at a low hydrogen supply rate.

Hereinafter, the present invention will be described in detail with reference to the accompanying drawings.

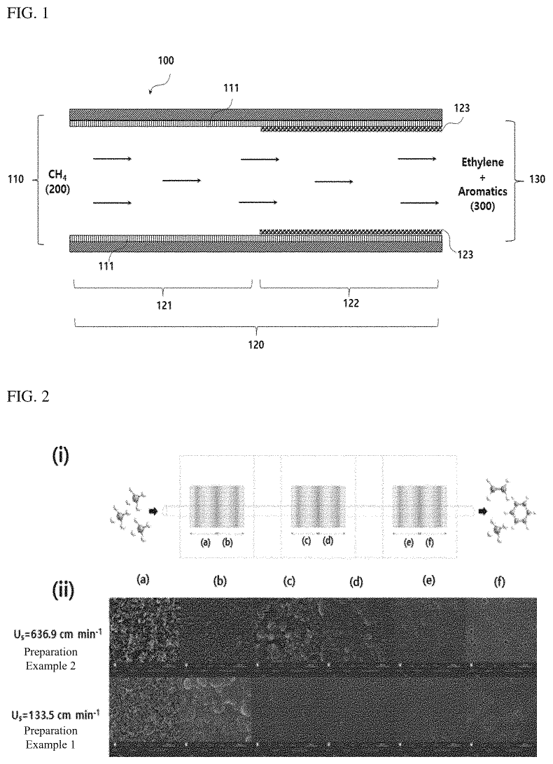

FIG. 1 is a longitudinal sectional view of a nonoxidative methane direct converting reactor according to an exemplary embodiment of the present invention, and referring to FIG. 1, a nonoxidative methane direct converting reactor 100 according to an exemplary embodiment of the present invention includes an inlet 110 through which a methane-containing supply 200 is introduced, a reactor 120 for producing an ethylene and aromatic compound-containing product 300 produced by a reaction of the methane-containing supply introduced from the inlet 110, and an outlet 130 for discharging the ethylene and aromatic compound-containing product 300 produced from the reactor 120.

The reactor 100 may have variable dimensions or shapes depending on production capacity, supply quantity, and catalyst, and it can be adjusted through various methods known to those skilled in the art. Preferably, the reactor is tubular in shape, with an inlet 110 formed on one side for introducing a methane-containing feedstock 200. The introduced methane-containing supply 200, while on the opposite side of the inlet, an outlet 130 is formed to discharge the reaction-completed ethylene and aromatic compound-containing product 300 to the outside or the reactor rear end.

In this case, the inlet 110 of the reactor may be disposed in the reactor without limitation, such as an upper side, a lower side, a right side, or a left side, and the outlet 130 may also be disposed at the other side of the inlet to correspond to the inlet.

The methane-containing supply 200 introduced into the inlet 110 may be used without limitation as long as it is a mixture including methane, and may include, for example, natural gas, and the like, and preferably, may include an inert gas and/or a non-inert gas in addition to methane.

The methane contained in the methane-containing supply may be 60% (v/v) or less, more preferably 18% (v/v) to 45% (v/v), based on the total volume of the methane-containing supply supplied into the reactor, and the inert gas and/or the non-inert gas may be 40% (v/v) or more, more preferably 55% (v/v) or more, based on the total volume of the methane-containing supply.

The inert gas may be nitrogen, helium, neon, argon, krypton, the non-inert gas may be carbon monoxide, hydrogen, carbon dioxide, water, monohydric alcohol (carbon number 1-5), dihydric alcohol (carbon number 2˜5), alkanes (carbon number 2-8), and the inert gas and the non-inert gas may be nitrogen, hydrogen, oxygen, water, and the like.

Meanwhile, when hydrogen is contained in addition to methane, a volume ratio of hydrogen to methane (H₂/CH₄) may be 1.1 to 5.0, preferably 1.1 to 4.5. If the volume ratio of hydrogen to methane (H₂/CH₄) is less than 1.1, a high partial pressure of the aromatic compound in the product may be caused, and as a result, a problem in which an operation range (temperature, pressure, fluid flow, etc.) of the reactor for suppressing the cokes formation reaction is narrowed may occur, and if the volume ratio of hydrogen to methane (H₂/CH₄) is greater than 5.0, a low reactivity due to a low partial pressure of methane may be caused, thereby increasing energy costs.

The first reaction zone unit gas hourly space velocity (GHSV) of the methane-containing supply may be 1,000 h⁻¹ to 6,000 h⁻¹. When the space velocity of the first reaction zone unit is less than 1,000 h⁻¹, there may be a problem in that the selectivity of cokes in the product is increased due to the acceleration of the C—C bonding reaction of the primary product, and when the space velocity of the first reaction zone unit is greater than 6,000 h⁻¹, there may be a problem in that the reactivity of the surface of the catalyst is lowered in the overall reactivity of the reactor, resulting in a decrease in reaction performance.

The methane-containing supply introduced into the reaction unit 120 from the inlet unit 110 passes through the first reaction zone unit 121 and the second reaction zone unit 122 to produce the ethylene and aromatic compound-containing product 300.

In the first reaction zone unit 121, the introduced methane-containing supply generates acetylene by thermal decomposition or nonoxidative methane direct converting reactor acetylene as shown in the following Reaction formula 1.

2CH₄→C₂H₂+3H₂ [Reaction Formula 1]

In this case, the reaction in the first reaction zone unit 121 may be performed at 900° C. to 1,300° C. and 10 bar or less, preferably 1,100° C. to 1,250° C. at 0.1 bar to 10 bar.

The above reaction condition range considers the selectivity and yield of hydrocarbons, and has the advantage of maximizing the selectivity of methane to hydrocarbons. That is, the cokes formation is minimized under the above conditions, so that the pressure drop due to the cokes formation during the reaction and the carbon efficiency due to the cokes formation can be minimized.

When the reaction temperature in the first reaction zone unit 121 is less than 900° C., the generation rate of radicals due to the activation of methane is low and thus the energy efficiency is low, and when the reaction temperature exceeds 1,300° C., the residence time of methane in the reactor should be minimized in order to suppress the generation of coke, and a large amount of energy required for heating the reactor may be required.

In addition, when the reaction pressure in the first reaction zone unit 121 exceeds 10 bar, cokes is accelerated to generate coke, and thus there may be a problem in that the reactor residence time and product cooling must be efficiently designed.

Meanwhile, the carbon layer 111 capable of causing a catalytic surface reaction in the nonoxidative methane direct conversion may be formed on the inner wall (inner circumferential surface) of the reactor in the first reaction zone unit 121. The carbon layer 111 is not a filled structure but a structure coated on the inner circumferential surface of the reactor, and is very advantageous in the formation of laminar flow and may improve stagnant flow of reactants and products. Consequently, the present invention can increase the reaction rate by acting as a catalyst surface during the activation of methane and at the same time can suppress the production of crystalline cokes due to additional reaction progress.

The carbon layer 111 may be a cokes layer formed by a nonoxidative methane direct converting reaction in a reactor. The carbon layer formed by the nonoxidative methane direct converting reaction may have a shape or a structure changed according to the direction of the axis of the reactor according to the reactivity of methane, thereby affecting methane activity and additional reactivity. In particular, when the surface roughness of the formed carbon layer is increased, since a high specific surface area is provided, a reaction in which radicals generated during the reaction are converted into cokes may be promoted. Therefore, it is necessary to design a carbon layer that can selectively increase the reaction rate of methane activation.

In this case, the nonoxidative methane direct converting reaction for forming a carbon layer in the reactor may be applied without limitation as long as it is a reaction condition for forming a cokes layer on the inner wall of the reactor, and for example, may be 0.1 bar to 10 bar at 900° C. to 2,000° C., may have a gas linear velocity of methane-containing inlet gas of 133 cm·min⁻¹ to 637 cm·min⁻¹, and may have a gas space velocity of 178 h⁻¹ to 849 h⁻¹. The methane-containing inlet gas may be the same as or different from the above-described methane-containing supply, and may further include an inert gas and/or a non-inert gas.

As described above, methane-containing supply is introduced to the second reaction zone unit 122 as acetylene is produced by the reaction in the first reaction zone unit 121, and acetylene-containing reactant introduced to the second reaction zone unit 122 is hydrogenated by hydrogen as shown in the following Reaction Formula 2 to be synthesized with ethylene and an aromatic compound. In this case, the hydrogen may be introduced as a methane-containing supply together with the methane through the inlet 110, or may be additionally supplied to the second reaction zone unit 122 through a hydrogen supply pipe (not shown).

C₂H₂+H₂→C₂H₄

3C₂H₂→C₆H₆

5C₂H₂→C₁₀H₈+H₂ [Reaction Formula 2]

In this case, the hydrogenation in the second reaction zone unit 122 may be performed at 30° C. to 900° C. and 10 bar or less, preferably 50° C. to 300° C. at 0.1 bar to 5 bar, the second reaction zone unit gas hourly space velocity (GHSV) may be 1.00×10⁴ mlg_{Pd}h⁻¹ to 1.00×10⁹ mlg_{Pd}h⁻¹, preferably 1.00×10⁷ mlg_{Pd}h⁻¹ to 1.00×10⁹ mlg_{cat}h⁻¹, and the mean residence time of the second reaction zone unit may be 0.01 sec to 5.6 sec.

The reaction conditions consider the selectivity and yield of hydrocarbons, and there is an advantage of maximizing the selectivity of acetylene to hydrocarbons. That is, cokes formation is minimized under the above conditions, so that pressure drop due to cokes formation during reaction and carbon immersion due to cokes formation can be minimized.

When the reaction temperature in the second reaction zone unit 122 is less than 30° C., there may be a problem in that the size of the reactor in the second reaction zone unit 122 should be increased compared to the first reaction zone unit 121 due to low reactivity of acetylene, and when the reaction temperature exceeds 900° C., side reaction (dehydrogenation and coupling) of acetylene predominates, and as a result, cokes formation may be promoted.

In addition, when the reaction pressure in the second reaction zone unit 122 exceeds 10 bar, side reaction (dehydrogenation and coupling) and hydrogenation reactivity of acetylene may be increased, such that cokes production may be promoted and catalyst performance may be deteriorated due to heat generation.

In addition, when the weight hourly space velocity (WHSV) in the second reaction zone unit 122 is less than 1.00×10⁹ mlg_{Pd}h⁻¹ or the mean residence time is more than 5.6 sec, there may be a problem that cokes production is accelerated, and when the is more than 1.00×10⁴ mlg_{Pd}h⁻¹ or the mean residence time is less than 0.01 sec, there may be a problem that acetylene reactivity is low and the size of the reactor should be increased compared to the first reaction zone unit.

In the second reaction zone unit 122, a carbon layer 111 which is the same as or different from the carbon layer formed in the first reaction zone unit may be formed on the inner circumferential surface (inner wall) of the reactor of the second reaction zone unit, and a metal compound 123 capable of promoting the aromatization of acetylene may be supported on the formed carbon layer 111.

The carbon layer, on which the metal compound is supported, formed on the inner circumferential surface of the reactor of the second reaction zone unit, is very advantageous in forming a laminar flow of the reactants and products, alleviates the stagnant flow of the reactants and products, and minimizes heat generation when acetylenes, which is an exothermic reaction, are hydrogenated while suppressing the generation of crystalline cokes due to the strong adsorption of the reactants on the surface of the catalyst cluster.

The carbon layer 111 of the second reaction zone unit may be formed on the inner circumferential surface of the reactor of the second reaction zone unit by the same method as that formed on the inner circumferential surface of the reactor of the first reaction zone unit, and the metal compound 123 on the carbon layer may be applied without limitation as long as it is a method capable of supporting the metal compound on the carbon layer such as a dry impregnation method, a wet impregnation method, or the like.

In this case, the metal compound 123 supported on the carbon layer may be palladium, platinum, iridium, rhodium, iron, chromium, nickel, molybdenum, gold, silver, copper, indium, or the like, may be preferably a metal including palladium and palladium in terms of acetylene hydrogenation activity, and may be less than 5 wt % based on the total weight of the carbon layer and the metal compound in the second reaction zone unit. When the supported ratio of the metal compound within the above range is 5 wt % or more, the selective acetylene hydrogenation reaction may be vigorously performed, such that it is difficult to control heat generation.

The carbon layer 111 of the first reaction zone unit and the second reaction zone unit has methane conversion activation energy ranging from 300 kJ/mol to 380 kJ/mol. The methane conversion activation energy is calculated by using an Arrhenius Equation, and when the methane conversion activation energy of the carbon layer is less than 300 kJ/mol, a secondary side reaction (C—C coupling reaction) of the primary product may be promoted, and as a result, a problem of increasing cokes selectivity in the product may occur, and when the methane conversion activation energy of the carbon layer is greater than 380 kJ/mol, a problem of reducing a catalyst surface having excellent performance in methane activation may occur, and as a result, the volume of the reactor may increase.

In addition, the carbon layer 111 of the first reaction zone unit and the second reaction zone unit may be formed to have a thickness of 1 micrometer to 1 mm. When the thickness of the carbon layer is less than 1 micrometer, the reactant reacts with the reactor surface, not the carbon layer, to increase cokes selectivity in the product, and when the thickness of the carbon layer exceeds 1 mm, the degree of curvature of the surface of the carbon layer increases to act as a scavenger of radicals generated under the reaction conditions, thereby increasing cokes selectivity in the product.

Meanwhile, in the nonoxidative methane direct converting reactor 100 according to the present invention, the ratio of the mean residence time of the first reaction zone unit to the above-described second reaction zone may be 1 to 30, preferably 3 to 30.

When the ratio of the mean residence time of the first reaction zone unit to the second reaction zone unit is less than 1, there may be an issue of increased reactor size in the second reaction zone compared to the first reaction zone, leading to a reduction in thermal efficiency. When the ratio is greater than 30, there may be a problem with low acetylene reactivity in the product, resulting in the presence of acetylene in the product.

The ethylene and aromatic compound-containing product synthesized as described above is discharged to the outer circumference or the reactor rear end through the outlet 130 of the reactor.

In another aspect, the present invention relates to a preparation method of ethylene and aromatic compound, wherein ethylene and an aromatic compound are prepared from methane using the nonoxidative methane direct converting reactor.

In the preparation method of ethylene and aromatic compound according to the present invention, when the methane-containing supply 200 is supplied through the inlet 110 of the nonoxidative methane direct converting reactor 100 described above, acetylene is generated by reaction in the first reaction zone unit 121 of the reactor, the generated acetylene is introduced into the second reaction zone unit 122, the acetylene introduced into the second reaction zone unit 122 is hydrogenated by hydrogen to prepare ethylene and an aromatic compound, and the ethylene and the aromatic compound are discharged through the outlet 130.

Since the preparation method of ethylene and aromatic compound according to the present invention is the same as described in the nonoxidative methane direct converting reactor corresponding thereto, the skilled person in the art can clearly understand the method, and thus the description thereof will be omitted in order to avoid redundancy.

Hereinafter, the present invention will be described in more detail through specific examples. The following Examples are only examples for helping understanding of the present invention, and the scope of the present invention is not limited thereto.

### Preparation Example 1 and Preparation Example 2

In order to form a carbon layer on the inner wall of the reactor, methane-containing inlet gas was introduced into a quartz tubular reactor (inner diameter: 4 mm, length: 45 cm) at the gas linear velocity and space velocity of Table 1, and a nonoxidative methane direct converting reaction was performed at 1230° C. with 1 bar [P_{CH4} (0.18 bar)] for 10 hours. In this case, the methane-containing inlet gas was used by mixing methane, hydrogen, and argon, and the volume ratio of H2/(CH₄+Ar) was 4, and the volume ratio of methane and argon was fixed at 9:1. After completing the nonoxidative methane direct converting reaction, the carbon layer formed in the axial direction of the reactor was measured by using SEM (MIRA3 LMU, Tescan) in order to confirm the carbon layer (thickness: 0.03 mm) formed on the inner wall of the reactor, and the result is shown in FIG. 2.

TABLE 1

| | Gas linear | Gas space |
|---|---|---|
| Category | velocity (cm · min⁻¹) | velocity (h⁻¹) |
| Preparation Example 1 | 133.5 | 178 |
| Preparation Example 2 | 636.9 | 848.8 |

As shown in FIG. 2, in both Preparation Examples 1 and 2, a porous cokes layer was formed at the inlet side of the reactor, and a cokes layer having a shape of a cobble stone was formed toward the outlet side of the reactor. It was confirmed that the shape of the cokes layer, that is, the carbon layer formed according to the axial direction of the reactor was different.

### Example 1 to 4

Pd(NO₃)₂·2H₂O (Sigma Aldrich, 99%) was mixed with an acetone solution to prepare a Pd solution so that the Pd concentration was 0.01 to 0.1 M. The prepared Pd solution was put into the reactor of Preparation Example 2 and maintained for 10 minutes, and then dried in an oven at 110° C. Thereafter, in order to remove Pd supported outside the second reaction zone, a reactor other than the reactor in which the carbon layer was formed was cut, and then the reactor of Preparation Example 2 under the same conditions was bonded at 1,650° C. or higher using a propane/oxygen torch. In order to reduce the supported Pd, a reactor was prepared by performing heat treatment while 100 sccm of 100% H₂ gas was flowed at 300° C. (10° C./min) for 1 hour. A total length of the prepared reactor was 45 cm, a length of the first reaction zone was 30 cm, and a length of the second reaction zone was 15 cm, and a content of Pd supported on a carbon layer of the second reaction zone unit was shown in Table 2.

TABLE 2

| Category | Example 1 | Example 2 | Example3 | Example4 |
|---|---|---|---|---|
| Pd content (μm | 0.134 | 0.214 | 1.503 | 3.655 |
| ol) | | | | |

### Comparison Example 1

After 15 g of Quartz particle was heated to 1700° C. at 10° C./min. in air and melted for 6 hours, the melted melt was cooled to obtain cristobalite (CRS). The obtained CRS was physically pulverized to have a particle size of 381˜864 μm, and then wet-impregnated with a Pd solution to prepare a Pd/CRS catalyst.

At this time, Pd(NO₃)₂·2H₂O (Sigma Aldrich, 99%) was used as the Pd precursor, and 0.5 wt % of the Pd precursor based on the weight of the CRS was added to 30 g of distilled water together with 6 g of the CRS support. The distilled water used here may have 5 weight ratio of distilled water/CRS. The mixture was stirred at 120 rpm for 6 hours at a temperature of 60° C., and water was removed using a rotary evaporator. Thereafter, the CRS carrying Pd, from which water was removed, was dried in an oven at 110° C. overnight, heated to 550° C. at 4° C./min in an air atmosphere, and then maintained for 4 hours to perform heat treatment. The heat-treated Pd-supported CRS was further heated to 300° C. at 10° C./min., and then heat-treated in a reducing atmosphere of 100% H₂ for 1 hour to prepare a catalyst. In this case, the loading amount of Pd is 28.188 μmol.

### Experimental Example 1: Activation Energy Measurement

In order to measure the methane reactivity according to the formation of the carbon layer formed on the inner circumferential surface of the reactor, the direct conversion reaction of methane was performed using the reactor prepared in Preparation Example 2. In this case, the methane-containing supply was used by mixing methane, hydrogen, and argon, and the volume ratio of H₂/(CH₄+Ar) was 4, and the volume ratio of methane and argon was fixed at 9:1. The reaction temperature of the direct conversion reaction of methane was 1180 to 1230° C., and the reaction pressure (Pₜₒₜₐₗ) was 1 bar.

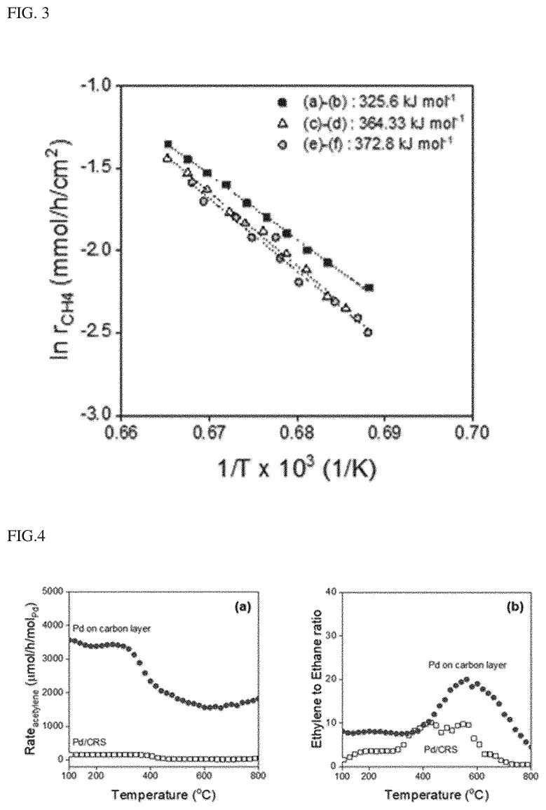

The vapor-phase hydrocarbon obtained after the reaction was analyzed by using 7820A GC of Agilent. The gaseous product was analyzed by Thermal conductivity detector (TCD) connected to a Shincarbon ST 80/100 column and Flame ionization detector (FID) detector connected to a RTx-VMS column, respectively. Hydrogen, methane, argon, ethane, ethylene, and acetylene were separated on a Shincarbon ST 80/100 column and detected by TCD, and the conversion rate was calculated by the area of methane compared to the area of argon, which is internal standard. The hydrocarbons and aromatic compound with over C₃ were separated by a RTx-VMS column and detected by FID. All gases were quantified using a standard sample. Cokes selectivity was calculated by [S_{coke}=100-Σ product selectivity]. Methane conversion rate and cokes selectivity were converted at the reaction rate according to the reaction temperature. This value was substituted into the Arrhenius Equation to calculate apparent activation energy (Ea), and the results are shown in FIG. 3.

As shown in FIG. 3, in the reactor in which the carbon layer was formed, the activation energy (Ea) of the carbon layer formed at the positions (a) and (b) of FIG. 1 was 325.6 kJmol⁻¹, the activation energy (Ea) of the carbon layer formed at the positions (c) and (d) was 364.33 kJmol⁻¹, and the activation energy (Ea) of the carbon layer formed at the positions (e) and (f) was 372.8 kJmol⁻¹. Accordingly, it was confirmed that the carbon layer formed in the reactor of Preparation Example 2 was formed to be suitable for hydrocarbon generation from methane.

### Experimental Example 3: Reactivity Measurement According to Pd Content

Acetylene hydrogenation reactivity of the methane conversion-derived product was measured in the reactors of Examples 1 to 4 prepared according to the Pd content. In this case, the methane-containing supply was used by mixing methane, hydrogen, and argon, and the volume ratio of H₂/(CH₄+Ar) was 4, and the volume ratio of methane and argon was fixed at 9:1. The nonoxidative methane converting reaction in the first reaction zone unit was conducted at a reaction pressure (Pₜₒₜₐₗ) of 1 bar at 1230° C., and the hydrogenation reaction of the acetylene in the second reaction zone unit was conducted at a reaction pressure (Pₜₒₜₐₗ) of 1 bar at 170° C. to 200° C. When the nonoxidative methane converting reaction was performed, the product composition was Methane 12.487 vol %, Ar 1.929 vol %, H₂ 83.457 vol %, C₂H₂ 1.063 vol %, C₂H₄ 0.912 vol %, C₂H₆ 0.052 vol %, C₃˜C₄ 0.020 vol %, Benzene 0.071 vol %, Toluene 0.004 vol %, Naphthalene 0.001 vol %, and alkyl aromatics 0.004 vol %.

The gaseous hydrocarbon obtained after the reaction was analyzed using 7820A GC. The gaseous product was analyzed by Thermal conductivity detector (TCD) connected to the Shincarbon ST 80/100 column and Flame ionization detector (FID) detector connected to the RTx-VMS column. Hydrogen, methane, argon, ethane, ethylene, and acetylene were separated on a Shincarbon ST 80/100 column and detected by TCD, and the conversion rate was calculated by the area of acetylene compared to the area of argon at internal standard. The hydrocarbons and aromatic compound with over C₃ were separated by a RTx-VMS column and detected by FID. All gases were quantified using a standard sample. The activation energy and the Ethylene/ethane ratio in the product according to the Pd content of each catalyst are shown in Table 3 below.

TABLE 3

| | Apparent activation | Ethylene/ethane ratio |
|---|---|---|
| Category | energy (Ea), KJ/mol | (at 200° C.) |
| Example 1 | 9.5 | 7.3 |
| Example 2 | 24.6 | 11.3 |
| Example 3 | 36.0 | 6.1 |
| Example 4 | 21.3 | 8.1 |

As shown in Table 3, it was confirmed that the activation energy was not significantly changed according to the content of Pd, which is a metal compound, and the ethylene ratio to ethane was also well maintained.

### Experimental Example 4: Reactivity Measurement According to Reaction Temperature

In order to measure reactivity according to reaction temperature, acetylene hydrogenation reactivity of a methane conversion-derived product in the reactor of Example 3 and Comparative Example 1 was measured. In this case, the methane-containing supply was used by mixing methane, hydrogen, and argon, and the volume ratio of H₂/(CH₄+Ar) was 4, and the volume ratio of methane and argon was fixed at 9:1. The nonoxidative methane converting reaction in the first reaction zone unit was conducted at a reaction pressure (Pₜₒₜₐₗ) of 1 bar at 1,230° C., and the hydrogenation reaction of the acetylene in the second reaction zone unit was conducted at a reaction pressure (Pₜₒₜₐₗ) of 1 bar at 100° C. to 800° C. When the nonoxidative methane converting reaction was performed, the product composition was Methane 12.487 vol %, Ar 1.929 vol %, H₂ 83.457 vol %, C₂H₂ 1.063 vol %, C₂H₄ 0.912 vol %, C₂H₆ 0.052 vol %, C₃˜C₄ 0.020 vol %, Benzene 0.071 vol %, Toluene 0.004 vol %, Naphthalene 0.001 vol %, and alkyl aromatics 0.004 vol %. The gas hourly space velocity was measured by performing the reaction at 2,544 h⁻¹ based on the second reaction zone, which is an acetylene conversion reaction zone, and the reaction activity was measured while raising the temperature of the second reaction zone to 5° C./min.

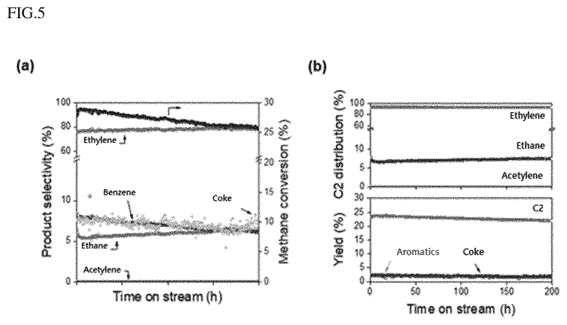

The vapor-phase hydrocarbon obtained after the reaction was analyzed using 7820A GC of Agilent. The gaseous product was analyzed by Thermal conductivity detector (TCD) connected to the Shincarbon ST 80/100 column and Flame ionization detector (FID) detector connected to the RTx-VMS column. Hydrogen, methane, argon, ethane, ethylene, and acetylene were separated on a Shincarbon ST 80/100 column and detected by TCD, and the conversion rate was calculated by the area of acetylene compared to the area of argon at internal standard. The hydrocarbons and aromatic compound with over C₃ were separated by a RTx-VMS column and detected by FID, and all gases were quantified using a standard sample. The ethylene and acetylene selectivities were calculated except for the contents included in the nonoxidative methane converting reaction product which is the reactant, and the methane conversion rate and cokes selectivity were calculated by converting them at the reaction rate according to the reaction temperature, and the acetylene conversion rate and ethane/ethylene selectivity according to the reaction temperature are shown in FIG. 4.

As shown in FIG. 4, it could be confirmed that acetylene was not aromatized at 100° C. to 800° C. and was selectively hydrogenated in the reactor of Example 3, and it could be confirmed that the acetylene conversion rate was significantly higher than that of Comparative Example 1, and the over-hydrogenation toward ethane was reduced at the ratio of the product. In addition, in the reactor of Example 3, it was confirmed that the methane-derived acetylene was widely converted to 100° C. to 800° C.

### Experimental Example 5: Reactivity Measurement According to H₂/CH₄+Ar Ratio

In order to measure the reactivity according to the ratio of methane, hydrogen, and argon contained in the methane-containing supply, a nonoxidative methane direct converting reaction for preparing ethylene was performed in the reactor of Example 3. The volume ratio of H₂/(CH₄+Ar) in the methane-containing supply was adjusted to 1-4 and then fed to the reactor of Example 3 to perform the reaction, in which the volume ratio of CH₄ and Ar was fixed at 9:1 and the reaction experiment was performed according to the gas hourly space velocity (GHSV). The nonoxidative methane converting reaction in the first reaction zone unit was conducted at a reaction pressure (Pₜₒₜₐₗ) of 1 bar at 1,230° C., and the hydrogenation reaction of the acetylene in the second reaction zone unit was conducted at a reaction pressure (Pₜₒₜₐₗ) of 1 bar at 200° C.

The vapor-phase hydrocarbon obtained after the reaction was analyzed by using 7820A GC of Agilent. The gaseous product was analyzed by Thermal conductivity detector (TCD) connected to a Shincarbon ST 80/100 column and Flame ionization detector (FID) detector connected to a RTx-VMS column, respectively. Hydrogen, methane, argon, ethane, ethylene, and acetylene were separated on a Shincarbon ST 80/100 column and detected by TCD, and the conversion rate was calculated by the area of methane compared to the area of argon, which is internal standard. The hydrocarbons and aromatic compound with over C₃ were separated by a RTx-VMS column and detected by FID, and all gases were quantified using a standard sample. Coke selectivity was calculated by [S_{coke}=100-Σ, product selectivity], and methane conversion and product and cokes selectivity are shown in Table 4 below.

TABLE 4

| | Volume ratio of H2/(CH4 + Ar) of a | | | |
|---|---|---|---|---|
| | methane-containing supply | | | |
| Category | 1 | 2 | 3 | 4 |
| GHSV (h−1) of the first reaction zone unit | 5,732 | 2,388 | 1,592 | 1,273 |
| WHSV (ml gPd h−1) of the second | 3.15 × 108 | 1.12 × 108 | 7.49 × 107 | 5.99 × 107 |
| reaction zone unit | | | | |
| Methane conversion (%) | 28.0 | 31.6 | 30.6 | 27.8 |
| Ethylene yield (mol C %) | 17.6 | 22.0 | 22.8 | 22.1 |
| Ethane selectivity (mol C %) | 3.0 | 3.4 | 4.4 | 5.8 |
| Ethylene selectivity (mol C %) | 63.0 | 69.6 | 74.7 | 79.4 |
| Acetylene selectivity (mol C %) | 0.3 | 0 | 0 | 0 |
| C3-C4 selectivity (mol C %) | 3.2 | 2.2 | 1.7 | 1.5 |
| Benzene selectivity (mol C %) | 17.7 | 14.4 | 11.2 | 8.3 |
| Naphthalene (mol C %) | 3.5 | 2.5 | 1.7 | 1.0 |
| Alkyl aromatics selectivity (mol C %) | 4.0 | 4.6 | 2.6 | 2.6 |
| Coke selectivity (mol C %) | 5.3 | 3.3 | 3.7 | 1.4 |

As shown in Table 4, it could be confirmed that even when the hydrogen content in the methane-containing supply was decreased, the reduction in the methane conversion rate and the ethylene yield was not large, and the cokes selectivity was not significantly increased.

### Experimental Example 6: Reaction Stability Evaluation According to Reaction Time

In order to evaluate the reaction stability according to the reaction time, a nonoxidative methane converting reaction for preparing ethylene was performed in the reactor of Example 3. Methane-containing supply adjusted the volume ratio of H₂/(CH₄+Ar) to 4 and supplied to the reactor of Example 3 to perform the reaction, in which the volume ratio of CH₄ and Ar was fixed at 9:1, and the flow rate of the methane-containing supply was 80 ml·min⁻¹. The nonoxidative methane converting reaction in the first reaction zone unit was conducted at a reaction pressure (Pₜₒₜₐₗ) of 1 bar at 1,230° C., and the hydrogenation reaction of the acetylene in the second reaction zone unit was conducted at a reaction pressure (Pₜₒₜₐₗ) of 1 bar at 200° C.

The vapor-phase hydrocarbon obtained after the reaction was analyzed by using 7820A GC of Agilent. The gaseous product was analyzed by Thermal conductivity detector (TCD) connected to a Shincarbon ST 80/100 column and Flame ionization detector (FID) detector connected to a RTx-VMS column, respectively. Hydrogen, methane, argon, ethane, ethylene, and acetylene were separated on a Shincarbon ST 80/100 column and detected by TCD, and the conversion rate was calculated as the area of methane compared to the area of argon at internal standard. The hydrocarbons and aromatic compound with over C₃ were separated by a RTx-VMS column and detected by FID. All gases were quantified using a standard sample. Coke selectivity was calculated by [S_{coke}=100-Σ product selectivity], and methane conversion and selectivity for 200 hours are shown in FIG. 5.

As shown in FIG. 5, it was confirmed that the reactor of Example 3 maintained a high conversion rate of methane for 200 hours, an average yield of ethylene was 21.2%, an average yield of C₂ was 22.82%, an average yield of Aromatics was 2.66%, an average yield of Coke was 1.40%, and acetylene was not produced.

Therefore, it could be confirmed that the nonoxidative methane direct converting reactor according to the present invention maximizes the catalytic reaction rate, minimizes cokes formation, and provides a high conversion rate of methane and a high yield of ethylene and an aromatic compound in the preparation of ethylene and an aromatic compound.

The present invention has been described with reference to the above-described embodiments and the accompanying drawings, but may constitute different embodiments within the concept and scope of the present invention. Accordingly, the scope of the present invention is defined by the appended claims and equivalents, and is not limited by the specific embodiments described herein.

1. A nonoxidative methane direct converting reactor, comprising:
an inlet through which a methane-containing supply is introduced; a reaction unit reacting the methane-containing supply introduced from the inlet to produce an ethylene and aromatic compound-containing product; and
an outlet through which the ethylene and aromatic compound-containing product produced from the reaction unit is discharged, wherein the reaction unit is divided into:
a first reaction zone unit producing acetylene by reaction of the methane-containing supply introduced from the inlet; and
a second reaction zone unit hydrogenating the acetylene produced from the first reaction zone unit to produce ethylene and an aromatic compound, wherein a carbon layer is formed on inner circumferential surfaces of the first reaction zone unit and the second reaction zone unit, and the second reaction zone unit, on which the carbon layer is formed, has a metal compound supported on the carbon layer.

2. The nonoxidative methane direct converting reactor of claim 1, wherein the carbon layer is a cokes layer formed by a nonoxidative methane direct converting reaction.

3. The nonoxidative methane direct converting reactor of claim 1, wherein the carbon layer has methane conversion activation energy of 300 kJ/mol to 380 kJ/mol.

4. The nonoxidative methane direct converting reactor of claim 1, wherein the metal compound comprises at least one selected from the group consisting of palladium, platinum, iridium, rhodium, iron, chromium, nickel, molybdenum, gold, silver, copper and indium.

5. The nonoxidative methane direct converting reactor of claim 1, wherein the methane-containing supply includes methane and hydrogen, and a volume ratio of hydrogen to the methane (H₂/CH₄) in a range of 1.1 to 5.0.

6. The nonoxidative methane direct converting reactor of claim 1, wherein the reaction of the first reaction zone unit is performed at a temperature of 900° C. to 1, 300° C. and 10 bar or less.

7. The nonoxidative methane direct converting reactor of claim 1, wherein the hydrogenation of the second reaction zone is performed at temperatures ranging from 30° C. to 900° C. and pressures below 10 bar.

8. The nonoxidative methane direct converting reactor of claim 1, wherein the first reaction zone unit has a gas hourly space velocity (GHSV) of 1,000 h⁻¹ to 6,000 h⁻¹.

9. The nonoxidative methane direct converting reactor of claim 1, wherein the second reaction zone weight hourly space velocity (WHSV) is 1.00×10⁴ mlg_{Pd}h⁻¹ to 1.00×10⁹ mlg_{Pd}h⁻¹.

10. A preparation method of ethylene and aromatic compound, the method comprising preparing ethylene and aromatic compound from methane using the nonoxidative methane direct converting reactor of claim 1.
